SEQUENCE LISTING (4 sequences)

SEQ ID NO: 1 - DNA, 20 bp, Artificial
  Forward primer of Mycobacterium tuberculosis complex (TB145U)
cgatcgctgatccggccaca

SEQ ID NO: 2 - DNA, 25 bp, Artificial
  Reverse primer of Mycobacterium tuberculosis complex (TB145L)
gcgtcggtgacaaaggccacgtagg

SEQ ID NO: 3 - DNA, 83 bp, Artificial
  IC molecule (TB145IC)
ctgatccggccacatatcgcgtttatgcgaggtcgggtgggcgggtcgttagtttcgttttgggcctacgtggcctttgtcac

SEQ ID NO: 4 - DNA, 1360 bp, Mycobacterium tuberculosis
  misc_feature: gene sequence encoding IS6110, an IS-like element of M.tuberculosis (NCBI accession number X17348)
cgatgaaccgccccggcatgtccggagactccagttcttggaaaggatggggtcatgtcaggtggttcatcgaggaggtacccgccggagctgcgtgagcgggcggtgcggatggtcgcagagatccgcggtcagcacgattcggagtgggcagcgatcagtgaggtcgcccgtctacttggtgttggctgcgcggagacggtgcgtaagtgggtgcgccaggcgcaggtcgatgccggcgcacggcccgggaccacgaccgaagaatccgctgagctgaagcgcttagcggcgggacaacgccgaattgcgaagggcgaacgcgattttaaagaccgcgtcggctttcttcgcggccgagctcgaccggccagcacgctaattaacggttcatcgccgatcatcagggccaccgcgagggccccgatggtttgcggtggggtgtcgagtcgatctgcacacagctgaccgagctgggtgtgccgatcgccccatcgacctactacgaccacatcaaccgggagcccagccgccgcgagctgcgcgatggcgaactcaaggagcacatcagccgcgtccacgccgccaactacggtgtttacggtgcccgcaaagtgtggctaaccctgaaccgtgagggcatcgaggtggccagatgcaccgtcgaacggctgatgaccaaactcggcctgtccgggaccacccgcggcaaagcccgcaggaccacgatcgctgatccggccacagcccgtcccgccgatctcgtccagcgccgcttcggaccaccagcacctaaccggctgtgggtagcagacctcacctatgtgtcgacctgggcagggttcgcctacgtggcctttgtcaccgacgcctacgtcgcaggatcctgggctggcgggtcgcttccacgatggccacctccatggtcctcgacgcgatcgagcaagccatctggacccgccaacaagaaggcgtactcgacctgaaagacgttatccaccatacggataggggatctcagtacacatcgatccggttcagcgagcggctcgccgaggcaggcatccaaccgtcggtcggagcggtcggaagctcctatgacaatgcactagccgagacgatcaacggcctatacaagaccgagctgatcaaacccggcaagccctggcggtccatcgaggatgtcgagttggccaccgcgcgctgggtcgactggttcaaccatcgccgcctctaccagtactgcggcgacgtcccgccggtcgaactcgaggctgcctactacgctcaacgccagagaccagccgccggctgaggtctcagatcagagagtctccggactcaccggggcggttcacga

(19) United States
(12) Patent Application Publication
(10) Pub. No.: US 2012/0100545 A1
(43) Pub. Date: Apr. 26, 2012

(54) METHOD AND/OR PRIMERS FOR THE DETECTION OF MYCOBACTERIUM TUBERCULOSIS

(75) Inventors: Masafumi Inoue, Singapore (SG); Timothy Barkham, Singapore (SG)

(73) Assignees: TAN TOCK SENG HOSPITAL, Singapore (SG); AGENCY FOR SCIENCE, TECHNOLOGY AND RESEARCH, Connexis (SG)

(21) Appl. No.: 13/382,056

(22) PCT Filed: Jul. 5, 2010
(86) PCT No.: PCT/SG2010/000251

§ 371 Date: Jan. 3, 2012

(30) Foreign Application Priority Data
Jul. 3, 2009 (SG) 200904595-6

(51) Int. Cl. C12Q 1/68; C07H 21/04

(52) U.S. Cl. 435/6.11; 536/24.33; 536/24.32; 435/6.12

(57) ABSTRACT

The invention provides oligonucleotide(s) for simple, specific and/or sensitive test(s) for the presence of Mycobacterium tuberculosis. In particular, the present invention provides oligonucleotide(s) for test(s) for Mycobacterium tuberculosis. Kit(s) comprising the oligonucleotide(s) for use as probe(s) and/or primer(s) useful in the test(s) are also provided.

### FIELD OF THE INVENTION

The present invention relates to primer(s), probes as well as method(s) and kit(s) using such primer(s) and/or probes for the detection of the presence of Mycobacterium tuberculosis.

### BACKGROUND TO THE INVENTION

Tuberculosis (TB) is a chronic, infectious disease that is generally caused by infection with Mycobacterium tuberculosis or by one or more organisms of the Mycobacterium tuberculosis complex. TB remains a major worldwide health problem today with about eight million new cases and three million deaths each year. The incidence of TB in Singapore is 35-40 per 100,000 people in a year. That equates to 4-5 new cases each day. Despite the increased dissemination of TB, the diagnosis is difficult to establish. In particular, the immune-logical mechanisms by which M. tuberculosis maintains and multiplies within the host are poorly understood.

The sequencing of the M. tuberculosis genome has facilitated an enormous research effort to identify potential M. tuberculosis proteins that theoretically may be expressed by the organism. However, sequence data alone is insufficient to conclude that any particular protein is expressed in vivo by the organism, let alone during infection of a human or animal.

Infection with M. tuberculosis bacilli, or reactivation of a latent infection, induces a host response comprising the recruitment of monocytes and macrophages to the site of infection. As more immune cells accumulate a nodule of granulomata forms comprising immune cells and host tissue that have been destroyed by the cytotoxic products of macrophages. As the disease progresses, macrophage enzymes cause the hydrolysis of protein, lipid and nucleic acids resulting in liquefaction of surrounding tissue and granuloma formation. Eventually the lesion ruptures and the bacilli are released into the surrounding lung, blood or lymph system. Although the infection may be asymptomatic for a considerable period of time, the disease is most commonly manifested as an acute inflammation of the lungs, resulting in fever and a productive cough. If left untreated, M. tuberculosis infection may progress beyond the primary infection site in the lungs to any organ in the body and generally results in serious complications and death. It is thus essential to diagnose TB early in the infection.

Conventional techniques, such as microscopy for acid fast bacteria is quick and relatively cheap but suffers from lack of sensitivity, ranging from 40-60%, and a consequent poor negative predictive value. In populations with significant levels of non-tuberculous mycobacteria (NTM), such as patients with AIDS, a positive smear is devalued as it still cannot be confirmed if the positive smear is a result of TB or NTM. NTM, are mycobacteria which do not cause tuberculosis or Hansen's disease (also known as leprosy). Examples of NTM include but are not limited to M. leprae, M. avium, M. kansasii and the like.

Currently, latent infection is diagnosed in a non-immunized person by a TB skin test, which yields a delayed hypersensitivity type response to an extract made from M. tuberculosis. Those immunized for TB or with past-cleared infection will respond with delayed hypersensitivity parallel to those currently in a state of infection, so the test must be used with caution, particularly with regard to persons from countries where TB immunization is common. Tuberculin tests also have the disadvantage of producing false negatives, especially when the patient is co-morbid with sarcoidosis, Hodgkins lymphoma, malnutrition, or most notably active TB diseases. The interferon gamma release assays are far more specific than the Tuberculin skin test but in a population with high levels of latent TB the value of a ‘reactive’ result in a symptomatic patient is low and the predictive value of a negative result is not sufficient to exclude TB.

Culture is still the most sensitive method but despite great improvements with broth based methods the time to reporting a positive is too slow to help with the immediate management decisions. This is because, M. tuberculosis is a slow-growing organism in the laboratory (it may take 4 to 12 weeks for blood or sputum culture). It is common practice to use culture as the ‘Reference standard’. However, 2-3% of MTBC culture positive samples are false positive.

A complete medical evaluation for TB must include a medical history, a physical examination, a chest X-ray, microbiological smears, and cultures. It may also include a tuberculin skin test and a serological test. The interpretation of the tuberculin skin test depends upon the person's risk factors for infection and progression to TB disease, such as exposure to other cases of TB or immunosuppression. Even after all these tests, the result of the diagnosis of TB may still only be a probable result that sometimes may be inconclusive. Current diagnostic microbiological methods are thus insensitive, non-specific and slow.

### SUMMARY OF THE INVENTION

The present invention is defined in the appended independent claims. Some optional features of the present invention are defined in the appended dependent claims. In particular, the present invention addresses the problems above, and provides highly sensitive and specific oligonucleotides, fragments and/or derivatives thereof useful in a method of detecting M. tuberculosis in patient specimens more efficiently. The primers and/or probes may be sensitive and specific in the detection of TB and provide rapid and cost-effective diagnostic and prognostic reagents for determining infection by M. tuberculosis and/or disease conditions associated therewith. These primers provide a means for cheap, fast and more accurate TB testing.

According to an aspect, the present invention provides at least one isolated oligonucleotide comprising, consisting essentially of, or consisting of at least one nucleotide sequence of SEQ ID NO:1 or SEQ ID NO:2, fragment(s), derivative(s), mutation(s), or complementary sequence(s) thereof. The oligonucleotide may be capable of binding to and/or being amplified from Mycobacterium tuberculosis complex.

These primers may be used in nucleic acid amplification (NAA) tests which may be more sensitive than the conventional methods available for diagnosis of TB.

According to another aspect, the present invention provides at least one pair of oligonucleotides comprising at least one forward primer and at least one reverse primer, wherein the forward primer comprises, consists essentially of or consists of SEQ ID NO:1, fragment(s), derivative(s), mutation(s), or complementary sequence(s) thereof and the reverse primer comprises, consists essentially of or consists of SEQ ID NO:2, fragment(s), derivative(s), mutation(s), or complementary sequence(s) thereof.

According to another aspect, the present invention provides at least one set of oligonucleotides comprising a pair of oligonucleotides according to any aspect of the present invention and at least one probe.

According to a further aspect, the present invention provides at least one amplicon amplified from M. tuberculosis complex using at least one forward primer comprising, consisting essentially of or consisting of the nucleotide sequence of SEQ ID NO:1, fragment(s), derivative(s), mutation(s), or complementary sequence(s) thereof and at least one reverse primer comprising, consisting essentially of or consisting of the nucleotide sequence of SEQ ID NO:2 fragment(s), derivative(s), mutation(s), or complementary sequence(s) thereof.

According to one aspect, the present invention provides at least one method of detecting the presence of M. tuberculosis in a biological sample, the method comprising the steps of:
(a) providing at least one biological sample;
(b) contacting at least one oligonucleotide, pair of oligonucleotides or set of oligonucleotides according to any aspect of the present invention, with at least one nucleic acid in the biological sample, and/or with at least one nucleic acid extracted, purified and/or amplified from the biological sample; and
(c) detecting any binding resulting from the contacting in step (b) whereby the M. tuberculosis is present when binding is detected.

According to one aspect, the present invention provides at least one method of amplifying M. tuberculosis nucleic acid, wherein said method comprises carrying out a polymerase chain reaction using SEQ ID NO:1, fragment(s), derivative(s), mutation(s), or complementary sequence(s) thereof and SEQ ID NO:2 fragment(s), derivative(s), mutation(s), or complementary sequence(s) thereof.

According to another aspect, the present invention provides at least one kit for the detection of M. tuberculosis, the kit comprising at least one oligonucleotide, pair of oligonucleotides or set of oligonucleotides according to any aspect of the present invention.

According to a particular aspect, there are provided highly sensitive and specific primers, fragments and/or derivatives thereof useful in a method of PCR capable of detecting M. tuberculosis DNA in patient specimens. This test may be used to examine the specimens from patients with active pulmonary tuberculosis. The primers may be sensitive and specific. Further, at least one IC molecule may be included in each reaction to monitor the PCR performance.

As will be apparent from the following description, preferred embodiments of the present invention allow for an optimal use of the primers and/or probes for the sensitive and specific the detection of TB where desired. This and other related advantages will be apparent to skilled persons from the description below.

### BRIEF DESCRIPTION OF THE FIGURES

FIG. 1 is an illustration depicting the first step in sample selection and PCR results of the present invention (in-house) compared with conventional methods such as culture and smear.

FIG. 2 is a flowchart of the second step in sample selection where the results obtained for separating the selected samples from FIG. 1 is further divided to pulmonary and non-pulmonary samples.

FIG. 3 is a flowchart showing the third step involved in selecting the prospective samples to test the primers and probes of the present invention.

FIG. 4 is results of PCR amplification products with and without PCR additives. The PCR products were observed in ethidium bromide-stained agarose gels. A) No additive, B) DMSO, 5%, C) Formamide, 5% and D) Betaine, 1M. The concentrations are the final concentration of the additives in the tubes. Lanes: M: 100 by DNA ladder; NTC: non template control, 54-56: positive clinical samples; 69-71: negative clinical samples. The expected PCR product was 145 base pairs (bp). An IC with an expected size of 95 by was spiked in all samples.

FIG. 5 is a gel picture of PCR products with formamide at a concentration of 1-10%. The PCR products were made from 5 copies of a TB DNA clone per reaction and observed in an ethidium bromide-stained agarose gel. Lanes: M: 100 by DNA ladder; C: control with no formamide added; 1-10: Percentage of formamide. Expected PCR product was 145 bp. An IC with an expected size of 95 by was spiked in all samples.

FIG. 6 is a gel picture of the PCR amplification products with formamide at a concentration of A) 0%, B) 3% and C) 5% observed in ethidium bromide-stained agarose gels. Lanes: M: 100 by DNA ladder; 1: NTC; 2-4: TB DNA 1 copy, 3 copies, 5 copies per reaction respectively; 20, 23, 25: positive clinical samples, 24, 27, 51: negative clinical samples. Expected PCR product was 145 bp. An IC with an expected size of 95 by was spiked in all samples. Samples 20 and 23 contain genomic DNA in the sample.

### DETAILED DESCRIPTION OF THE PREFERRED EMBODIMENTS

Bibliographic references mentioned in the present specification are for convenience listed in the form of a list of references and added at the end of the examples. The whole content of such bibliographic references is herein incorporated by reference.

### DEFINITIONS

The term “biological sample” is herein defined as a sample of any tissue and/or fluid from at least one animal and/or plant. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. Environmental samples include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to the methods disclosed herein. In particular, a biological sample may be of any tissue and/or fluid from at least a human being.

The term “complementary” is used herein in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” The degree of complementarity between nucleic acid strands has significant effects on the efficiency and strength of hybridization between nucleic acid strands. This is of particular importance in amplification reactions, as well as detection methods that depend upon binding between nucleic acids. In particular, the “complementary sequence” refers to an oligonucleotide which, when aligned with the nucleic acid sequence such that the 5′ end of one sequence is paired with the 3′ end of the other, is in “anti-parallel association.” Certain bases not commonly found in natural nucleic acids may be included in the nucleic acids disclosed herein and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. Where a first oligonucleotide is complementary to a region of a target nucleic acid and a second oligonucleotide has complementary to the same region (or a portion of this region) a “region of overlap” exists along the target nucleic acid. The degree of overlap may vary depending upon the extent of the complementarity.

The term “comprising” is herein defined as “including principally, but not necessarily solely”. Furthermore, the term “comprising” will be automatically read by the person skilled in the art as including “consisting of”. The variations of the word “comprising”, such as “comprise” and “comprises”, have correspondingly varied meanings.

The term “derivative,” is herein defined as the chemical modification of the oligonucleotides of the present invention, or of a polynucleotide sequence complementary to the oligonucleotides. Chemical modifications of a polynucleotide sequence can include, for example, replacement of hydrogen by an alkyl, acyl, or amino group.

The term “fragment” is herein defined as an incomplete or isolated portion of the full sequence of an oligonucleotide which comprises the active/binding site(s) that confers the sequence with the characteristics and function of the oligonucleotide. In particular, it may be shorter by at least one nucleotide or amino acid. More in particular, the fragment comprises the binding site(s) that enable the oligonucleotide to bind to M. tuberculosis complex. In particular, the fragment of the forward primer may comprise at least 10, 12, 15, 18 or 19 consecutive nucleotides of SEQ ID NO:1 and/or the reverse primer may comprise at least 10, 12, 15, 18, 19, 20, 22, or 24 consecutive nucleotides of SEQ ID NO:2. More in particular, the fragment of the primer may be at least 15 nucleotides in length.

The term “internal control (IC) molecule” is herein defined as the in vitro transcribed oligonucleotide molecule which is co-amplified by the same primer set for M. tuberculosis used in the method of the present invention. In particular, the IC may be mixed in the reaction mixture to monitor the performance of PCR to avoid false negative results. The probe to detect this IC molecule may be specific to the interior part of this molecule. This interior part may be artificially designed and may not occur in nature.

The term “Mycobacterium tuberculosis complex” as used in the context of the invention means one or more organisms such as M. tuberculosis, M. bovis, M. africanum, M. canetti, M. microti and the like.

The term “mutation” is herein defined as a change in the nucleic acid sequence of a length of nucleotides. A person skilled in the art will appreciate that small mutations, particularly point mutations of substitution, deletion and/or insertion has little impact on the stretch of nucleotides, particularly when the nucleic acids are used as probes. Accordingly, the oligonucleotide(s) according to the present invention encompasses mutation(s) of substitution(s), deletion(s) and/or insertion(s) of at least one nucleotide. Further, the oligonucleotide(s) and derivative(s) thereof according to the present invention may also function as probe(s) and hence, any oligonucleotide(s) referred to herein also encompasses their mutations and derivatives.

The term “nucleic acid in the biological sample” refers to any sample that contains nucleic acids (RNA or DNA). In particular, sources of nucleic acids are biological samples including, but not limited to blood, saliva, cerebral spinal fluid, pleural fluid, milk, lymph, sputum and semen.

According to one aspect, the present invention provides at least one isolated oligonucleotide comprising or consisting of at least one nucleotide sequence of SEQ ID NO:1 or SEQ ID NO:2, fragment(s), derivative(s), mutation(s) or complementary sequence(s) thereof. The oligonucleotide may be capable of binding to and/or being amplified from Mycobacterium tuberculosis complex. The Mycobacterium tuberculosis complex may be selected from the group consisting of M. tuberculosis, M. bovis, M. africanum, M. canetti, and M. microti. These primers may bind to one or more of M. tuberculosis, M. bovis, M. africanum, M. canetti, or M. microti. These primers may be sensitive and specific to Mycobacterium tuberculosis complex and not to NTM.

The primers according to any aspect of the present invention may be used to distinguish the genotype of M. tuberculosis from M. africanum, M. bovis, M. microti, and M. canettii. The M. tuberculosis genotype may comprise a drug-resistant strain of M. tuberculosis. The drug resistant strain of M. tuberculosis may be resistant to one or more drugs selected from the group consisting of: rifampin, ethambutol, isoniazid, diarylquinolone, fluoroquinolone, streptomycin and pyrazinamine. The drug resistant strain of M. tuberculosis may be a multi-drug resistant strain which may be resistant to a plurality of drugs selected from the group consisting of: rifampin, ethambutol, isoniazid, diarylquinolone, fluoroquinolone, streptomycin and pyrazinamide.

The oligonucleotide sequence may be between 13 and 35 linked nucleotides in length and may comprise at least 70% sequence identity to SEQ ID NO:1 or SEQ ID NO:2. A skilled person will appreciate that a given primer need not hybridize with 100% complementarity in order to effectively prime the synthesis of a complementary nucleic acid strand in an amplification reaction. A primer may hybridize over one or more segments such that intervening or adjacent segments are not involved in the hybridization event, (e.g., for example, a loop structure or a hairpin structure). In particular, the sequence of the oligonucleotide may have 80%, 85%, 90%, 95% or 98% sequence identity to SEQ ID NO:1 or SEQ ID NO:2.

An extent of variation of 70% to 100%, or any range therewithin, of the sequence identity is possible relative to the specific primer sequences disclosed. Determination of sequence identity is described in the following example: a primer 20 nucleotides in length which is identical to another 20 nucleotides in length primer having two non-identical residues has 18 of 20 identical residues ( 18/20=0.9 or 90% sequence identity). In another example, a primer 15 nucleotides in length having all residues identical to a 15 nucleotides segment of primer 20 nucleobases in length would have 15/20=0.75 or 75% sequence identity with the 20 nucleotides primer.

Percent homology, sequence identity or complementarity, can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). A skilled person is able to calculate percent sequence identity or percent sequence homology and able to determine, without undue experimentation, the effects of variation of primer sequence identity on the function of the primer in its role in priming synthesis of a complementary strand of nucleic acid for production of an amplification product.

According to another aspect, the present invention provides at least one pair of oligonucleotides comprising at least one forward primer and at least one reverse primer, wherein the forward primer comprises, consists essentially of or consists of SEQ ID NO:1, fragment(s), derivative(s), mutation(s), and complementary sequence(s) thereof and the reverse primer comprises, consists essentially of or consists of SEQ ID NO:2, fragment(s), derivative(s), mutation(s), and complementary sequence(s) thereof.

According to another aspect, the present invention provides at least one set of oligonucleotides comprising a pair of oligonucleotides according to any aspect of the present invention and at least one probe.

The probe may be labeled with a fluorescent dye at 5′ and 3′ ends thereof. Examples of the 5′-labeled fluorescent dye may include, but are not limited to, 6-carboxyfluorescein (FAM), hexachloro-6-carboxyfluorescein (HEX), tetrachloro-ó-carboxyfluorescein, and Cyanine-5 (Cy5). Examples of the 3′-labeled fluorescent dye may include, but are not limited to, 5-carboxytetramethylrhodamine (TAMRA) and black hole quencher-1,2,3 (BHQ-1,2,3).

The oligonucleotide according to any aspect of the present invention may be used in a method for the detection of M. tuberculosis from either a clinical or a culture sample, wherein the clinical samples may be selected from sputum, bronchoalveolar lavage fluid, pleural fluid, ascetic/peritoneal fluid, cerebrospinal fluid (CSF), pus, faecal matter, urine, amniotic fluid, menstrual blood, peripheral blood or other body fluids, lymph node, pus or other aspirate and tissue biopsies.

According to a further aspect, the present invention provides at least one amplicon amplified from M. tuberculosis complex using at least one forward primer comprising the nucleotide sequence of SEQ ID NO:1 and at least one reverse primer comprising the nucleotide sequence of SEQ ID NO:2.

According to one aspect, the present invention provides at least one method of detecting the presence of M. tuberculosis complex in a biological sample, the method comprising the steps of:
(a) providing at least one biological sample;
(b) contacting at least one oligonucleotide, pair of oligonucleotides or set of oligonucleotides according to any aspect of the present invention, with at least one nucleic acid in the biological sample, and/or with at least one nucleic acid extracted, purified and/or amplified from the biological sample; and
(c) detecting any binding resulting from the contacting in step (b) whereby the M. tuberculosis complex is present when binding is detected.

The method may be used for determining the identity and quantity of M. tuberculosis in a sample comprising contacting the sample with a pair of primers according to any aspect of the present invention and a known quantity of a calibration polynucleotide comprising a calibration sequence, concurrently amplifying nucleic acid from the M. tuberculosis in the sample with the pair of primers and amplifying nucleic acid from the calibration polynucleotide in the sample with the pair of primers to obtain a first amplification product comprising a M. tuberculosis identifying amplicon and a second amplification product comprising a calibration amplicon, obtaining molecular mass and abundance data for the M. tuberculosis identifying amplicon and for the calibration amplicon wherein the 5′ and 3′ ends of the M. tuberculosis identifying amplicon and the calibration amplicon are the sequences of the pair of primers or complements thereof, and distinguishing the M. tuberculosis identifying amplicon from the calibration amplicon based on their respective molecular masses, wherein the molecular mass of the M. tuberculosis identifying amplicon indicates the identity of the M. tuberculosis, and comparison of M. tuberculosis identifying amplicon abundance data and calibration amplicon abundance data indicates the quantity of M. tuberculosis in the sample.

According to one aspect, the present invention provides at least one method of amplifying M. tuberculosis nucleic acid, wherein said method comprises carrying out a polymerase chain reaction using SEQ ID NO:1 and SEQ ID NO:2.

The method according to any aspect of the present invention may further comprise a step of mixing an internal molecule (IC) and a probe specific to the IC with the biological sample. The IC may comprise the nucleotide sequence of SEQ ID NO:3. The use of the IC may improve the efficiency of the TB diagnosis increasing the accuracy of results.

The method according to any aspect of the present invention may be used in PCR amplification for specific diagnosis of tubercular meningitis, abdominal tuberculosis, gastrointestinal tuberculosis, genitourinary tuberculosis besides the pulmonary tuberculosis. Also the PCR amplification may be used to detect only active diseases and not the old exposures thus being more specific and accurate in the diagnosis.

According to another aspect, the present invention provides at least one kit for the detection of M. tuberculosis, the kit comprising at least one oligonucleotide, pair of oligonucleotides or set of oligonucleotides according to any aspect of the present invention.

It is submitted that the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

The evaluation of the diagnostic accuracy of the method according to the present invention containing an internal control (IC).

### Study Population

The evaluation was carried out on two sets of samples. The first set came from 414 original DNA extracts from one year of processing diagnostic requests for TB PCR at Tan Tock Seng Hospital (TTSH, Singapore); they represented a ‘retrospective’ group. All the DNA extracts were frozen at −80° C.

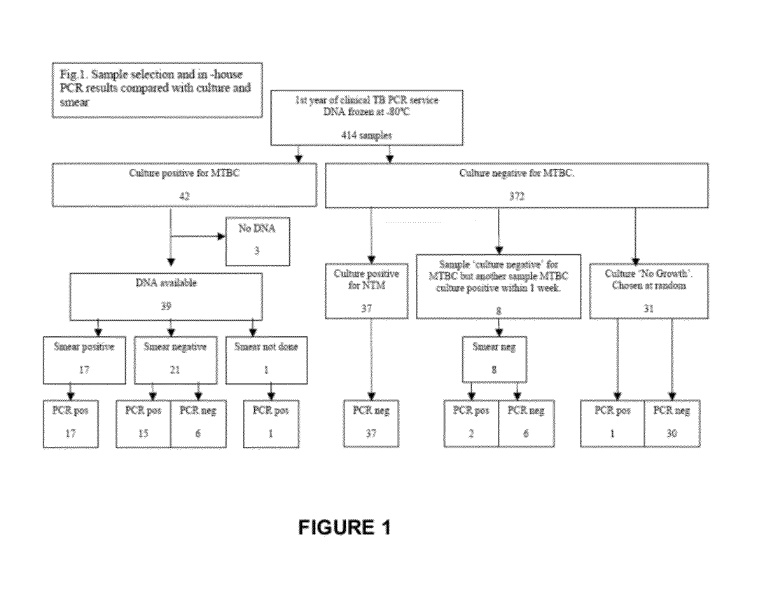

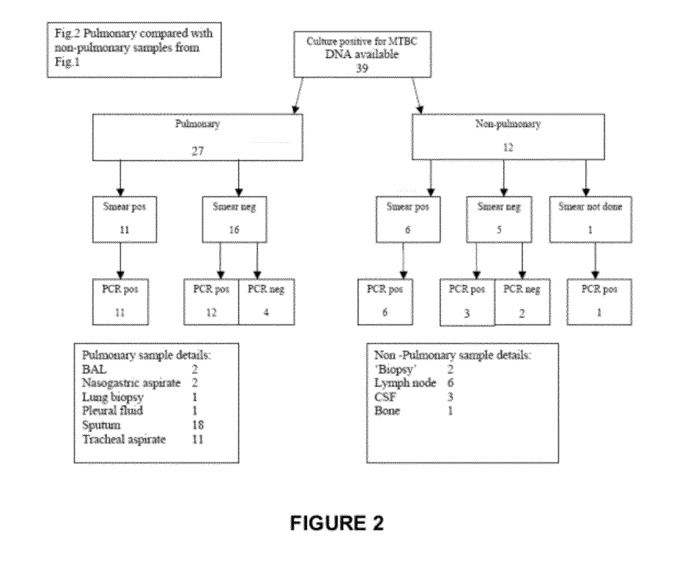

Samples from three sub-groups within the retrospective group were then selected as shown in FIGS. 1 and 2. All samples that had been reported to be culture positive for M. Tuberculosis complex (MTBC), all those reported to be culture negative for MTBC but culture positive for NTM and a random selection from those that were culture negative for both MTBC and NTM were selected.

A second set of samples was prospectively gathered by staff not involved with this evaluation who was asked to harvest aliquots from a mixture of smear positive and negative clinical samples being processed for mycobacterial smear and culture. The culture results were not known at the time of harvesting. The PCR was carried out by one experienced staff member without knowledge of the details of the samples or the patients' background. Data was made anonymous after collection.

### Sample Processing

Respiratory samples were liquefied with an equal volume of 1% N-acetyl cysteine, vortexed vigorously and left to stand for 15 mins. They were then centrifuged at 3000 rpm and the supernatant discarded. An aliquot of 1.5 mL of the deposit was stored at −80° C. as were uncentrifuged aliquots of cerebrospinal (CSF) and pleural fluids and aliquots of tissue/biopsy material that had been minced with 0.5 ml sterile saline.

DNA extraction was performed with the NucliSens easyMAG system (BIOMERIEUX, The Netherlands) with off board lysis. After the instrument had dispensed the lysis buffer, 500 ul of each specimen was added to the respective vessel in a Class II Biosafety cabinet and mixed well by pipetting up and down. Then 40 μl of QIAGEN Proteinase K was added to each vessel and mixed well by pipetting up and down. The mixture was incubated at room temperature for 30 min before being returned to the EasyMag instrument for automated extraction with a 25 μl elution protocol. The eluate was stored at −80° C.

### Primers

A set of primers used for amplification was derived from the gene sequence encoding IS6110, an IS-like element of M. tuberculosis (NCBI accession number X17348; SEQ ID NO:4). The sequences of forward primer (U) and reverse primer (L) are

(SEQ ID NO: 1)
TB145U: (5′-3′)CGATCGCTGATCCGGCCACA
and
(SEQ ID NO: 2)
TB145L: (5′-3′)GCGTCGGTGACAAAGGCCACGTAGG.

An internal control (IC) was mixed in the reaction mixture to monitor the performance of the PCR to detect false negative results. This IC molecule is co-amplified by the same primer set for TB indicated above. The interior part of this IC molecule was artificially designed and does not occur in nature. The sequence of IC molecule is:

TB145IC
(SEQ ID NO: 3)
(5′-3′)CTGATCCGGCCACATATCGCGTTTATGCGAGGTCGGGTGGGC
GGGTCGTTAGTTTCGTTTTGGGCCTACGTGGCCTTTGTCAC.

### Polymerase Chain Reaction (PCR)

PCR was performed with the Finnzyme Phire Hot Start DNA polymerase (catalog no. F-120) in a 25 μl reaction volume containing 5 μl of DNA sample, 100 copies of IC molecule, 1.25 μl of formamide at a final concentration of 5% and each primer at a final concentration of 0.3 μM in a thermal cycler. An Eppendorf Mastercycler-ep-gradient-S (Hamburg, Germany) was used with the following steps and conditions: initial activation at 98° C. for 65 sec, followed by 40 cycles of denaturation at 98° C. for 17 sec, annealing at 69° C. for 20 sec, and extension at 72° C. for 15 sec, and a final extension at 72° C. for 1 min. After amplification, the PCR products were analyzed by conventional gel electrophoresis. The PCR assay was designed and optimised at the Institute of Molecular and Cellular Biology, Singapore.

The reference standard for sensitivity analysis was defined as ‘MTBC culture positive’ as reported by an external TB laboratory using both solid and liquid based media as it is the most sensitive method available. For analysis of specificity, MTBC culture cannot be accepted as a single reference standard as samples submitted from patients on therapy, which renders samples culture negative, would mislead the analysis and overestimate the false positive rate. For example, it is possible to have MTBC culture negative samples that came from patients who had another recent sample reported as MTBC culture positive. As it is clearly unhelpful to treat then as real negatives for analysis purposes they were excluded from analysis but are clearly identified and presented in the results.

Samples found to be PCR positive but without any MTBC culture positive samples within 2 months were re-extracted and submitted to repeat PCR. The PCR products were sequenced to ascertain specificity. The sequencing was performed by an external laboratory and submitted to a BLAST analysis on the NCBI website. If the repeat PCR was positive and the sequence matched ‘MTBC’, then the results for these samples were not considered false positives so were excluded from the analysis of specificity. Similarly they were not included in the analysis of sensitivity.

Statistical analysis was performed on a web based program (www.quantitativeskills.com/sisa/statistics/diagnos.htm). Confidence intervals were only calculated for Set 1, the ‘clinically requested PCR samples’. They could not be calculated in cases of 100% sensitivity.

The reference standard method was routine mycobacterial culture performed at an external Laboratory within 24-72 hrs of sample collection as per the routine diagnostic workflow.

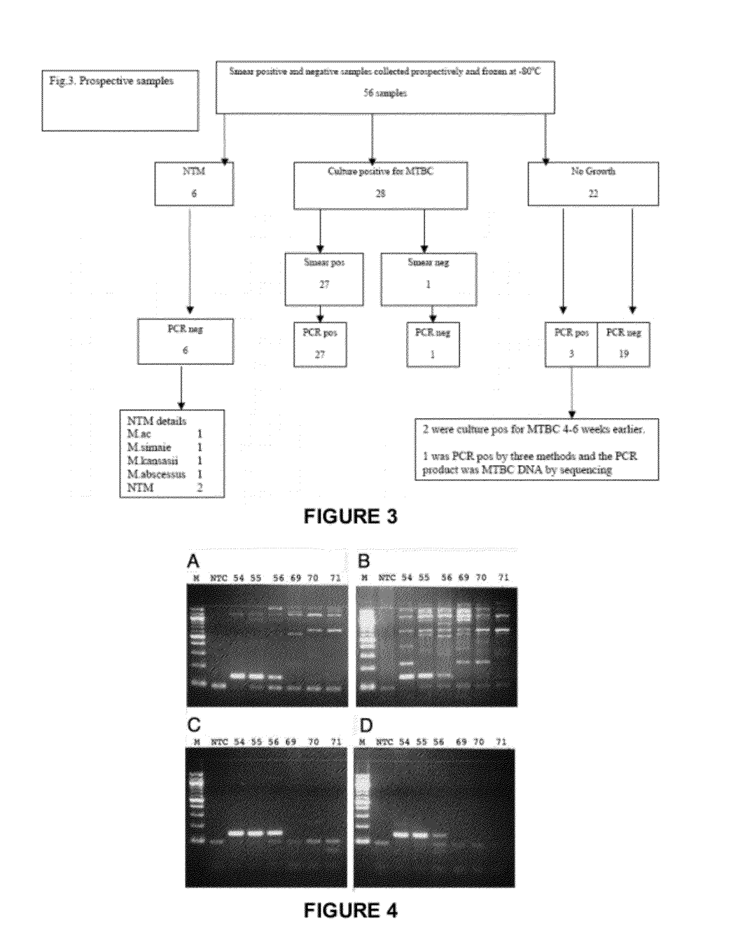

Details and results for the first set (samples from clinical PCR requests) are shown in FIG. 1 with the MTBC culture positive group further presented by specimen type in FIG. 2. The second set (prospectively harvested samples) is presented in FIG. 3. Analysis of sensitivity is shown in Table.1 and specificity in Table 2. Details of 6 samples with apparent false positive PCR results which were taught to be true positives are shown in Table 3. Four were from patients with MTBC isolated from other samples collected within one week (two cases) or 6 weeks (2 cases). Two PCR positive samples came from patients without any other samples MTBC culture positive. These were repeatedly PCR positive and the sequence of the PCR product in each case was a 100% match ( 145/145 bases) according to a BLAST analysis with numerous examples of M. tuberculosis on genebank (NCBI database). These patients may have had TB or the samples may have been contaminated, in either case TB DNA was present. The PCR system may be ‘clean’ as all the samples reported as positive with the diagnostic TB PCR over a year were also ‘culture positive’.

TABLE 1
Sensitivity of PCR compared with MTBC culture for Pulmonary
and non-pulmonary samples for clinically requested PCR (Set
1) and a prospectively gathered collection (Set 2).

| | Set 1 | | | Set 2 | |
|---|---|---|---|---|---|
| | No. PCR positive/ | Sensitivity | | No. PCR positive/ | Sensitivity |
| | No. MTBC positive | % (95% CI) | | No. MTBC positive | % |
| All samples | 33/39 | 85 | (73-96) | 27/28 | 96 |
| Smear positive | 17/17 | 100 | (—) | 27/27 | 100 |
| Smear negative | 15/21 | 71 | (52-91) | 0/1 | — |
| Smear not done | 1/1 | — | | — | — |
| Pulmonary | 23/27 | 85 | (72-99) | 26/27 | 96 |
| Smear positive | 11/11 | 100 | (—) | 26/26 | 100 |
| Smear negative | 12/16 | 75 | (54-96) | 0/1 | — |
| Non-pulmonary | 10/12 | 83 | (62-100) | 1/1 | — |
| Smear positive | 6/6 | 100 | (—) | 1/1 | — |
| Smear negative | 3/5 | 60 | (17-100) | — | — |

| | | | | |
|---|---|---|---|---|
| Smear not done | 1/1 | — | — | — |

TABLE 2
Specificity of PCR compared with culture for samples for clinically requested
PCR (Set 1) and a prospectively gathered collection (Set 2).

| | Set 1 | | Set 2 | |
|---|---|---|---|---|
| | No. PCR negative/ | Specificity | No. PCR negative/ | Specificity |
| | No. MTBC negative | % | No. MTBC negative | % |
| NTM culture positive | 38/38 | 100 | 6/6 | 100 |
| Culture negative | 29/29 | 100 | 19/19 | 100 |
| Samples excluded | | | | |
| Patient had MTBC in | 6/8 | — | 0/2 | — |
| another sample | | | | |
| Repeat PCR positive and | 0/1 | — | 0/1 | — |
| PCR product sequence is | | | | |
| ‘MTBC’ | | | | |

Note the samples excluded.

TABLE 3
Details of 6 PCR positive samples that were MTBC culture
negative but that we believe are true positives

| | | Patient previously | Time since last | Repeat | |
|---|---|---|---|---|---|
| Sample | | MTBC culture | MTBC culture | PCR | PCR product |
| Set No. | Sample type | positive | positive sample | result | sequence data |
| 1 | Pleural fluid | Yes | 1 week | Not done | Not done |
| 1 | Nasogastric | Yes | 1 week | Not done | Not done |
| | aspirate | | | | |
| 1 | Skin biopsy | No | Nil | Positive | 100% match |
| 2 | Sputum | Yes | 4 weeks | Not done | Not done |
| 2 | Sputum | Yes | 6 weeks | Not done | Not done |
| 2 | Sputum | No | Nil | Positive | 100% match |

For clinically requested PCR samples (Set.1), the sensitivity for samples positive by smear and culture was high at 100%. For smear negative but culture positive samples it varied from 60% to 75% for non-pulmonary and pulmonary samples respectively. The data for the prospectively collected samples (Set 2) was better for it did not reflect ‘clinically’ requested PCR, the sensitivity data was also less accurate than that from the samples in the Set 1, which were originally clinically requested for PCR. In this clinically requested group the overall sensitivity for both smear positive and smear negative samples was 85% compared with 96% in Set 2, the prospective group. This emphasizes the importance of evaluating performance on clinically requested samples.

The specificity depends on the definition of a true negative. Six samples were PCR positive but MTBC culture negative (Table 3). These were all excluded from the specificity analysis for they may be true positives, not false positives. Four were from patients with MTBC cultured from a previous sample. The remaining two were repeatedly PCR positive and the PCR products shown by sequencing to be MTBC. Excluding these six gives a specificity of 100%.

The results show 100% sensitivity in smear positive samples and the yield of 71% (95% CI=52-91%) from smear negatives. The IC proved useful as it detected inhibition in 10 samples in Set.1. They were retested at a ten fold dilution; four were PCR positive. This inhibition rate of 8.7% ( 10/115) was higher than expected compared with a previous commercial assay that showed <1% inhibition. This may be testimony to the well balanced IC and contributes to finding more true positives.

This work shows that the present assays can perform well and significantly outperform commercial assays.

### Example 2

The PCR protocol mentioned in Example 1 was used which yielded cleaner results and benefited from an integral internal control. This protocol used different enzymes that significantly shortened the turnaround time of the assay.

### Primers and IC Molecule

The primers and IC molecule which were mentioned in Example 1 were used. The primers were designed using the sequence of M. tuberculosis IS6110 element and direct repeat region, strain 191, NCBI accession number Y14048.

### PCR

PCR was performed with the Finnzymes Phire Hot Start DNA polymerase (catalog no. F-120) in a 25-μl reaction volume containing 5 μl of DNA sample, 100 copies of IC molecule and each primer at a final concentration of 0.6 μM in a thermal cycler. in our studies, Eppendorf Mastercycler-ep-gradient-S (Hamburg, Germany) was used with the following steps and conditions: initial activation at 98° C. for 65 sec, followed by 40 cycles of denaturation at 98° C. for 17 sec, annealing at 69° C. for 20 sec, and extension at 72° C. for 15 sec, and a final extension at 72° C. for 1 min. After amplification, the PCR products were analyzed by the conventional gel electrophoresis.

### PCR Additives

In the clinical evaluation significant non-specific amplification products were observed. A variety of additives and enhancing agents available to increase the yield, specificity and consistency of PCR reactions were tested. In particular, four PCR additives were tested in an attempt to eliminate non-specific priming: 5% DMSO (dimethyl sulfoxide), 5% formamide, 1M Betaine (N,N,N-trimethylglycine) and 100 mM TMAC (tetramethylammonium chloride). 5% formamide gave the best results as shown in FIG. 4. No product was detected with the addition of 100 mM TMAC so the data is not shown.

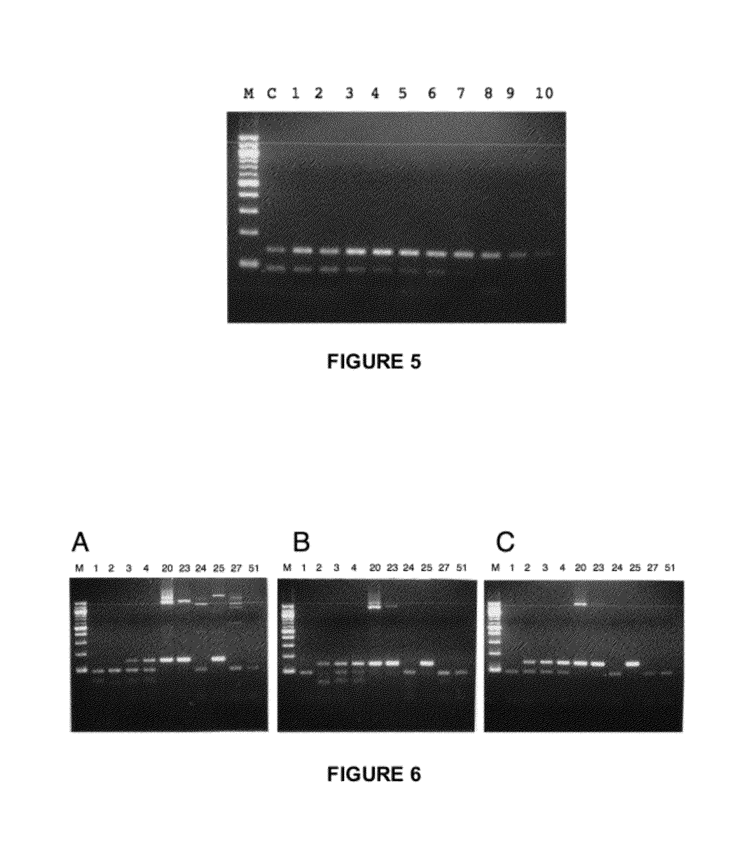

Formamide was tested, with the use of a TB DNA clone as template, across a range of final concentrations between 1-10%. The addition of formamide at a concentration of 1-8% yielded more product compared to the absence of an additive as shown in FIG. 5. When tested on clinical samples, formamide at a final concentration of 5% was better than 3% at eliminating non-specific priming as shown in FIG. 6.

### REFERENCES

Thierry, D., M. D. Cave, K. D. Eisenach, J. T. Crawford, J. H. Bates, B. Gicquel, and J. L. Guesdon. 1990. IS6110, an IS-like element of Mycobacterium tuberculosis complex. Nucleic Acids Res 18:188.
Updated Guidelines for the Use of Nucleic Acid Amplification Tests in the Diagnosis of Tuberculosis. MMWR Jan. 16, 2009/Vol. 58/No. 1/Pg. 7-10.
Eisenach, K. D., M. D. Cave, J. H. Bates, and J. T. Crawford. 1990. Polymerase chain reaction amplification of a repetitive DNA sequence specific for Mycobacterium tuberculosis. J Infect Dis 161:977-81.
Antonio Aceti,a Stefania Zanetti,b Maria S Mura,a Leonardo A Sechi,b Franco Turrini,c Franca Saba,a Sergio Babudieri,a Franca Mannu,c Giovanni Faddad. Identification of HIV patients with active pulmonary tuberculosis using urine based polymerase chain reaction assay. Thorax 1999; 54:145-146.
Gabriela Torrea, Philippe Van de Perre, Martial Ouedraogo, Alain Zougba, Adrian Sawadogo, Benoý\ddot{}t Dingtoumda, Boukari Diallo, Marie Christine Defer, Issiaka Sombie, Stefania Zanetti and Leonardo A. Sechi. PCR-based detection of the Mycobacterium tuberculosis complex in urine of HIV-infected and uninfected pulmonary and extrapulmonary tuberculosis patients in Burkina Faso. Journal of Medical Microbiology. 2005, 54, 39-44.
Irina Botezatu, Ol'ga Serdyuk, Galina Potapova, Valery Shelepov, Raisa Alechina, Yuriy Molyaka, Vitaliy Anan'ev, Igor Bazin, August Garin, Mehti Narimanov, Vasiliy Knysh, Hovsep Melkonyan, Samuil Umansky, and Anatoly Lichtenstein. Genetic Analysis of DNA Excreted in Urine: A New Approach for Detecting Specific Genomic DNA Sequences from Cells Dying in an Organism. Clinical Chemistry. 2000 46:8 1078-1084.
Mengjun Wang, Timothy M. Block, Laura Steel, Dean E. Brenner, and Ying-Hsiu Su. Preferential Isolation of Fragmented DNA Enhances the Detection of Circulating Mutated k-ras DNA. Clinical Chemistry 2004 50, No. 1, 211-213.
Sambrook and Russel, Molecular Cloning: A Laboratory Manual, Cold Springs Harbor Laboratory, New York (2001)

1. An isolated oligonucleotide comprising at least one nucleotide sequence of SEQ ID NO:1 or SEQ ID NO:2.

2. The oligonucleotide according to claim 1, wherein the oligonucleotide sequence is between 13 and 35 linked nucleotides in length and comprises at least 70% sequence identity to any one of SEQ ID NO:1 or SEQ ID NO:2.

3. The isolated oligonucleotide according to claim 1, consisting of SEQ ID NO:1, or SEQ ID NO:2.

4. The isolated oligonucleotide according to claim 1, wherein the oligonucleotide is capable of binding to and/or being amplified from Mycobacterium tuberculosis complex.

5. The isolated oligonucleotide according to claim 4, wherein the Mycobacterium tuberculosis complex is selected from the group consisting of M. tuberculosis, M. bovis, M. africanum, M. canetti, and M. microti.

6. A pair of oligonucleotides comprising at least one forward primer and at least one reverse primer, wherein the forward primer comprises SEQ ID NO:1 and the reverse primer comprises SEQ ID NO:2.

7. The pair of oligonucleotides according to claim 6, wherein the forward primer consists of SEQ ID NO:1, and the reverse primer consists of SEQ ID NO:2.

8. (canceled)

9. (canceled)

10. A method of detecting and/or quantitating Mycobacterium tuberculosis complex in a biological sample, the method comprising the steps of:
(a) providing at least one biological sample;
(b) contacting at least one oligonucleotide according to claim 1, with at least one nucleic acid in the biological sample, and/or with at least one nucleic acid extracted, purified and/or amplified from the biological sample; and
(c) detecting and/or quantitating any binding resulting from the contacting in step (b) whereby the Mycobacterium tuberculosis complex is present when binding is detected.

11. (canceled)

12. The method according to claim 10, further comprising the step of mixing an internal molecule (IC) and a probe specific to the IC with the biological sample after step (a).

13. The method according to claim 12, wherein the IC comprises the nucleotide sequence of SEQ ID NO:3.

14. (canceled)

15. (canceled)

16. A kit for the detection of Mycobacterium tuberculosis complex, the kit comprising at least one oligonucleotide according to claim 1.

17. A kit for the detection of Mycobacterium tuberculosis complex, the kit comprising at least one pair of oligonucleotides according to claim 6.

18. (canceled)
